(19) United States
(12) Patent Application Publication
(10) Pub. No.: US 2017/0128115 A1
(43) Pub. Date: May 11, 2017

(54) FIXTURE FOR INSTALLING LOCKING SCREWS INTO INTRAMEDULLARY NAILS

(71) Applicants: Sushil K. Dhawan, Poughkeepsie, NY (US); Aruna Bakhru Dhawan, Poughkeepsie, NY (US)

(72) Inventors: Sushil K. Dhawan, Poughkeepsie, NY (US); Aruna Bakhru Dhawan, Poughkeepsie, NY (US)

(21) Appl. No.: 15/299,345

(22) Filed: Oct. 20, 2016

Related U.S. Application Data
(60) Provisional application No. 62/253,703, filed on Nov. 11, 2015.

(51) Int. Cl. A61B 17/88; A61B 17/72

(52) U.S. Cl. CPC A61B 17/8872; A61B 17/725; A61B 2017/90

(57) ABSTRACT

An apparatus is described for aiding in installing locking screws in intramedullary nails when repairing bone fractures. The apparatus includes a long section, a short section, and a removable insert. The long section includes a long radiopaque rod running along a long axis. The short section joins the long section and defines a central channel running through it along a short axis that is substantially normal to the long axis. The removable insert is at least partially located inside the central channel and comprises a short radiopaque rod oriented along the short axis.

### BACKGROUND OF THE INVENTION

Intramedullary (IM) nailing is the standard of care for operative procedures dealing with long-bone fractures. These fractures include selected metaphyseal, shaft, and diaphyseal fractures in the femur, tibia, and humerus. The IM nailing procedure involves the use of an IM nail that is placed in the medullar cavity of the long bone and extends the length of the bone, as well as several additional screws. The combination of the IM nail and the screws serve to bear compressive, bending, and torsional loads in order to allow adequate healing of the long bone.

FIG. 1 shows a perspective view of a conventional IM nail 100, while FIGS. 2A-2E show diagrammatic representations of how the IM nail 100 may be installed to repair a severely fractured left femur. The IM nail 100 is an elongate rod of titanium with a set of proximal locking holes 105 and a set of distal locking holes 110 that pass through the IM nail 100 crosswise to its longitudinal axis. During installation, an incision 205 is made above the medullary cavity of a femur 210 (FIG. 2A) and a drill 215 is used to access that canal from the top (FIG. 2B). Subsequently the IM nail 100 is inserted into the length of the femur 210 (FIG. 2C). Once the IM nail 100 is so placed, holes are drilled transverse to the femur 210, and a proximal locking screw 220 and a distal locking screw 225 are screwed into the femur 210 such that the locking screws 220, 225 pass through two of the locking holes 105, 110 (FIGS. 2D and 2E). While just one proximal locking screw 220 and one distal locking screw 225 are shown in FIGS. 2D and 2E, more are frequently utilized in actual practice.

IM nails typically are built with proximal jigs that allow the surgeon to easily install one or more proximal locking screws. In contrast, installation of distal locking screws is typically not so easy. Such installation tends to be highly dependent on the use of a c-arm x-ray machine, which includes imaging equipment that may be rotated about the long bone to allow the bone and the installations to be imaged from various angles. Nevertheless, while drilling a hole for a distal locking screw, the drill and the surgeon's hands tend to block the x-ray imaging of precisely that locking hole in the IM nail that the surgeon is attempting to penetrate (hereinafter, the “target distal locking hole”). The drilling of the holes for the distal locking screws thereby becomes somewhat “blind.”

The most widely used technique for drilling a distal hole thereby becomes a “guess and check” technique in which the surgeon aligns the drill using the c-arm before making an incision, marks the position of the drill, makes the incision, and then inserts the drill into the incision. Once the drill is inserted into the incision, the surgeon is unable to ensure that the drill is properly aligned with the target distal locking hole as the drill blocks the x-ray. The surgeon then proceeds to mark the surface of the bone with the drill and then removes the drill to check the location of the mark relative to the target hole by x-ray. If the drill mark does not match the location of the target distal locking hole, the surgeon must adjust the drill and repeat the process. Due to the trial-and-error nature of this technique, the surgeon can spend an extended amount of time under x-ray, potentially causing prolonged x-ray exposure to both the surgeon and the patient. At the same time, the patient may be kept under anesthesia in the operating room for extended periods, which may be detrimental to the patient.

Several attempts have been made to address the above-identified disadvantages. Some methods for installing distal locking screws, for example, abandon the use of x-rays altogether in favor of magnetic detection. These techniques, however, frequently do not afford an appropriate amount of accuracy. Other techniques attempt to use distal jigs that are specific to certain long bones (i.e., they are not universal), are highly complex and costly, and require a lot of training to use properly. Because of these disadvantages, most surgeons choose to continue using the “guess and check” method.

For the foregoing reasons, there is a need for apparatus and methods that address the above-identified deficiencies.

### SUMMARY OF THE INVENTION

Embodiments of the present invention address the above-identified needs by providing a fixture to aid in installing locking screws in intramedullary nails when repairing long-bone fractures.

Aspects of the invention are directed to an apparatus comprising a long section, a short section, and a removable insert. The long section comprises a long radiopaque rod running along a long axis. The short section joins the long section and defines a central channel running therethrough along a short axis that is substantially normal to the long axis. The removable insert is at least partially disposed inside the central channel and comprises a short radiopaque rod oriented along the short axis.

Additional aspects of the invention are directed to a method of placing a locking screw into a locking hole of an intramedullary nail in a bone. An apparatus is obtained comprising a long section, a short section, and a removable insert. The long section comprises a long radiopaque rod running along a long axis. The short section joins the long section and defines a central channel running therethrough along a short axis that is substantially normal to the long axis. The removable insert is at least partially disposed inside the central channel and comprises a short radiopaque rod oriented along the short axis. While performing the method, the image of the short radiopaque rod is caused to appear centered over an image of the locking hole utilizing x-ray imaging.

Advantageously, embodiments in accordance with aspects of the invention provide a surgeon with a means of avoiding the “guess and check” method of fixing a distal locking screw, substantially increasing efficiency and reducing the time needed to perform the procedure, as well as several other advantages.

### BRIEF DESCRIPTION OF THE DRAWINGS

These and other features, aspects, and advantages of the present invention will become better understood with regard to the following description and accompanying drawings where:

FIG. 1 shows a perspective view of an IM nail;

FIGS. 2A-2E show diagrammatic representations of how the FIG. 1 IM nail may be installed in a broken femur;

FIG. 3A shows an exploded perspective view of a fixture in accordance with an illustrative embodiment of the invention;

FIGS. 3B-3D show magnified sectional views of the FIG. 3A fixture along the planes indicated in FIG. 3A;

FIG. 4 shows a partial sectional view of a portion of the FIG. 3A fixture with the removable insert inserted into the short section;

FIG. 5 shows a flow chart of an illustrative method for utilizing the FIG. 3A fixture to install a distal locking screw in an IM nail;

FIGS. 6A-6C show diagrammatic representations of how the FIG. 3A fixture may be utilized to install a locking screw into an IM nail by the FIG. 5 method;

FIGS. 7A-7C show diagrammatic representations of an x-ray display while performing the FIG. 5 method;

FIGS. 8A and 8B show diagrammatic representations of how the FIG. 3A fixture may be utilized to install a locking screw into an IM nail with a middle stabilizing rod;

FIG. 8C shows a magnified elevational view of a portion of a modified version of the FIG. 3A fixture with the middle stabilizing rod; and

FIG. 9 shows a partial sectional view of a portion of the FIG. 3A fixture with a modified removable insert inserted into the short section.

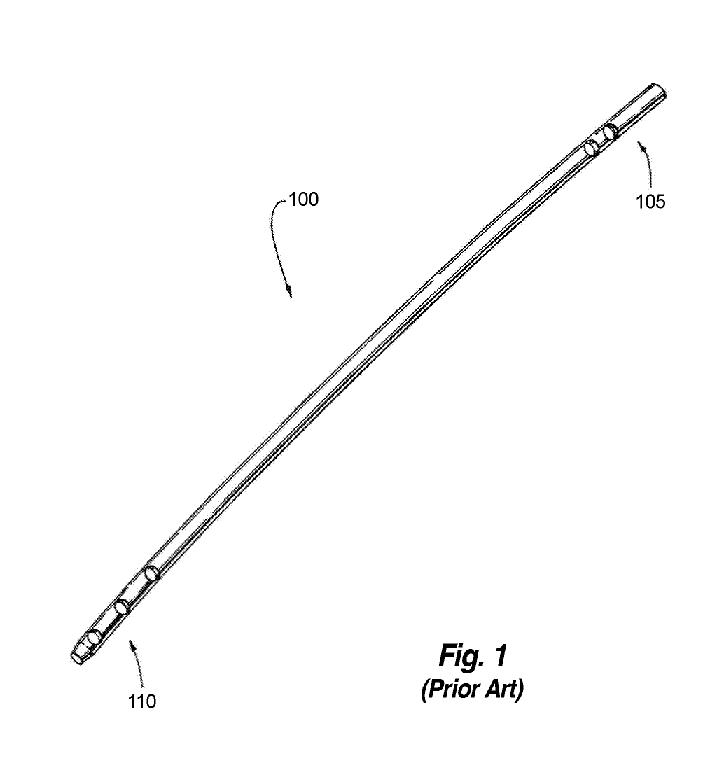

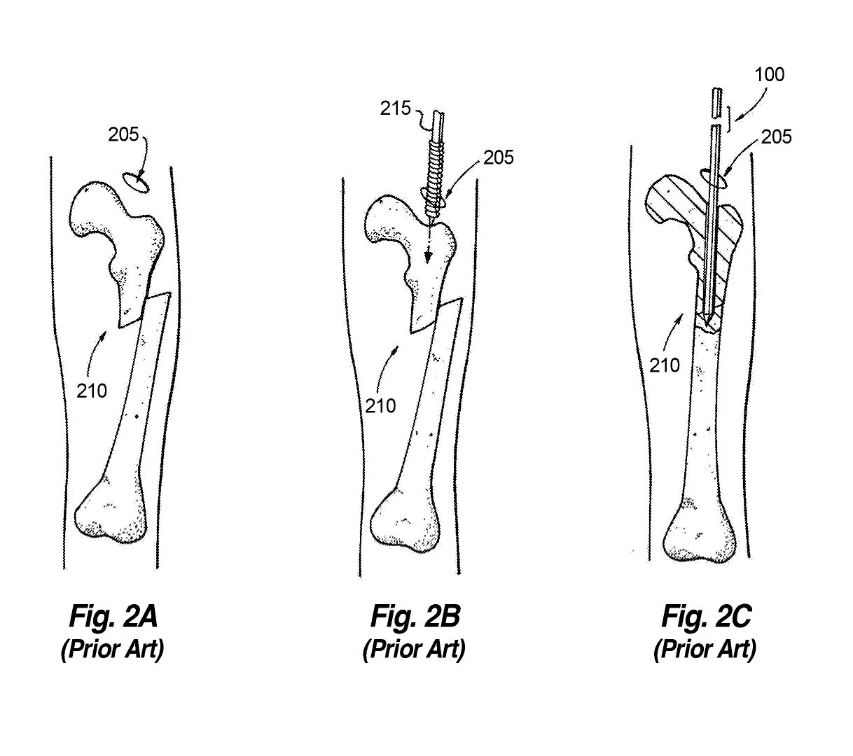

### DETAILED DESCRIPTION OF THE INVENTION

The present invention will be described with reference to illustrative embodiments. For this reason, numerous modifications can be made to these embodiments and the results will still come within the scope of the invention. No limitations with respect to the specific embodiments described herein are intended or should be inferred.

As used herein and in the appended claims, the words “substantially normal” are intended to mean within plus or minus fifteen degrees of normal. Additionally, as used herein, “x-ray imaging” includes any imaging method wherein an x-ray beam is passed through the body, the x-rays are either absorbed or scattered by the internal structures, and the remaining X-ray pattern is transmitted to a detector. “X-ray imaging” therefore includes, but is not limited to, radiography, fluoroscopy, and computed tomography (CT).

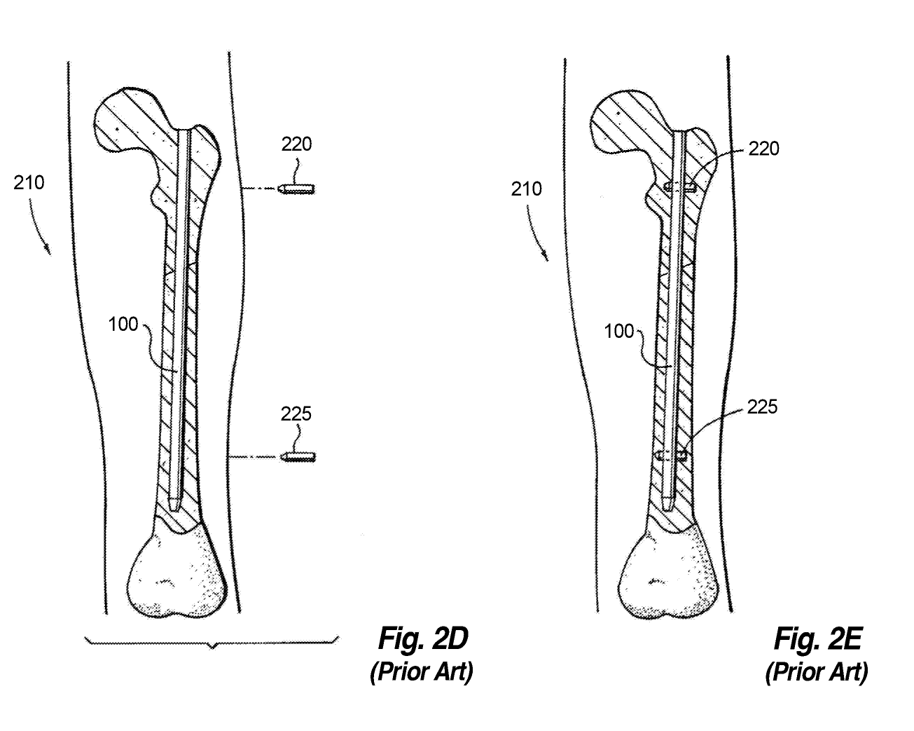

FIGS. 3A-3D show aspects of a fixture 300 in accordance with an illustrative embodiment of the invention for installing distal locking screws (also sometimes called “distal interlocking screws,” “distal locking bolts,” and “distal interlocking bolts”) into IM nails. FIG. 3A shows an exploded perspective view of the fixture 300, while FIGS. 3B-3D show magnified sectional views of the fixture 300 taken along the cleave planes indicated in FIG. 3A.

Conceptually, the fixture 300 may be separated into three main components: a long section 305, a short section 310, and a removable insert 315. The long section 305 comprises a long cylindrical body 320, while the short section 310 comprises a short cylindrical body 325. In the present embodiment, both the long cylindrical body 320 and the short cylindrical body 325 are transparent. One end of the long cylindrical body 320 joins one end of the externally-cylindrical short cylindrical body 325 so that the long and short sections 305, 310 combine to form an L shape. Fixed together in this manner, a longitudinal axis of the long section 305 (the long axis 327) is oriented so as to be substantially normal to a longitudinal axis of the short section 310 (the short axis 329).

The long section 305 and the short section 310 comprise several elements that aid in their functionality. The long cylindrical body 320 of the long section 305, for example, surrounds a long radiopaque rod 330, which runs along the long axis 327 for essentially the entire length of the long cylindrical body 320. The short cylindrical body 325 of the short section 310, in contrast, is hollow, and defines a central channel 335 running therethrough along the short axis 329. A radiopaque band 340 is supported by the short cylindrical body 325 about one-third of the way from the bottom of the short section 310 towards its top, and encircles a portion of the central channel 335. In the present embodiment, the radiopaque band 340 is circular. At the same time, the short section 310 terminates in a jagged edge 345 at an end opposite to where the short section 310 joins the long section 305. The short section 310 defines a short run of internal threads 347 near its top.

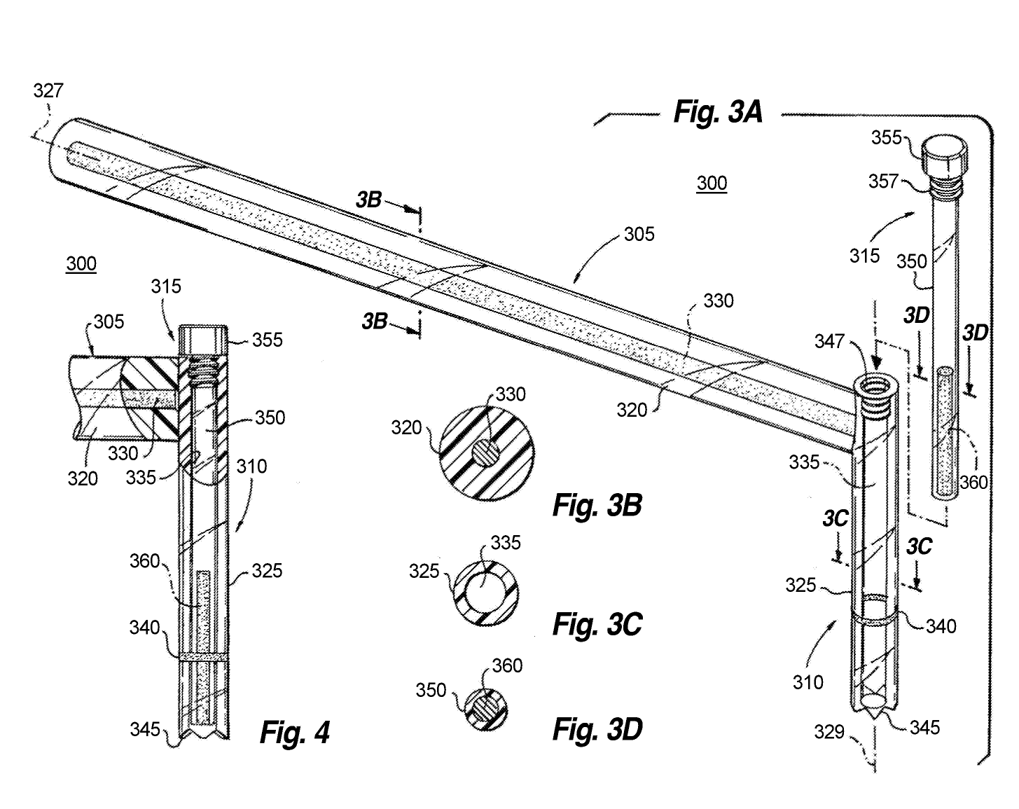

The removable insert 315 comprises a cylindrical insert body 350 topped by a wider cylindrical cap 355 that is too large to fit into the central channel 335. The cylindrical insert body 350 defines several external threads 357 near its top, complementary to the internal threads 347 on the short section 310. The cylindrical insert body 350 is dimensioned such that it may be removably inserted and ultimately threaded into the central channel 335 of the short section 310 to the point where the wider cylindrical cap 355 lays on a top of the short section 310 (i.e., the cap 355 abuts the short section 310). That is, the cylindrical insert body 350 has a diameter slightly smaller than the inner diameter of the central channel 335. FIG. 4 shows an elevational view of a portion of the fixture 300 with the removable insert 315 inserted into the central channel 335. Like the long section 305, the removable insert 315 contains a radiopaque rod. More particularly, the removable insert 315 comprises a short radiopaque rod 360 that runs along a longitudinal axis of the cylindrical insert body 350. In the present illustrative embodiment, the short radiopaque rod 360 occupies the bottom portion of the removable insert 315 and is cylindrical. When the removable insert 315 is inserted into the short section 310 in the manner shown in FIG. 4, the short radiopaque rod 360 is also oriented along the short axis 329.

For reasons that will be elucidated below, the long section 305 is preferably substantially longer than the short section 310. In one or more non-limiting embodiments, for example, the long section 305 may be about ten inches long, while the short section 310 may be about four inches long. Nevertheless, as is the case with all dimensions provided herein, these values are merely illustrative. In the present embodiment, the long section 305 also has a larger outside radius than the short section 310 to aid in handling the fixture 300. The central channel 335 of the short section 310 will ultimately be used as a guide for a drill bit used to create a distal hole for a distal locking screw, so the central channel 335 is preferably of large enough inner diameter to accommodate an appropriate drill bit. If, for example, a 4.0 mm-diameter three-fluted drill bit is utilized, the inside diameter of the central channel 335 may be just slightly larger than 4.0 mm.

With regard to materials, the long cylindrical body 320, the short cylindrical body 325, and the cylindrical insert body 350 (with the cylindrical cap 355) are preferably formed of a radiolucent material such as a plastic or the like. In contrast, as their names would suggest, the long radiopaque rod 330, the radiopaque band 340, and the short radiopaque rod 360 are preferably formed of a material that is opaque during x-ray imaging (e.g., during imaging by x-ray), such as a metal or the like. Alternatively, the radiopaque elements may be painted or coated in a radiopaque material.

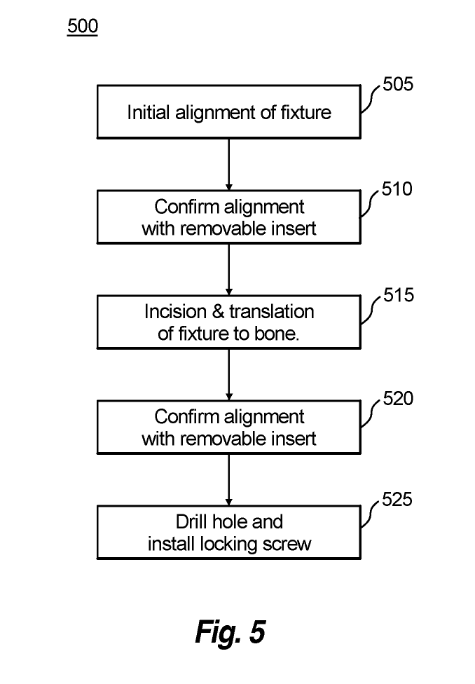

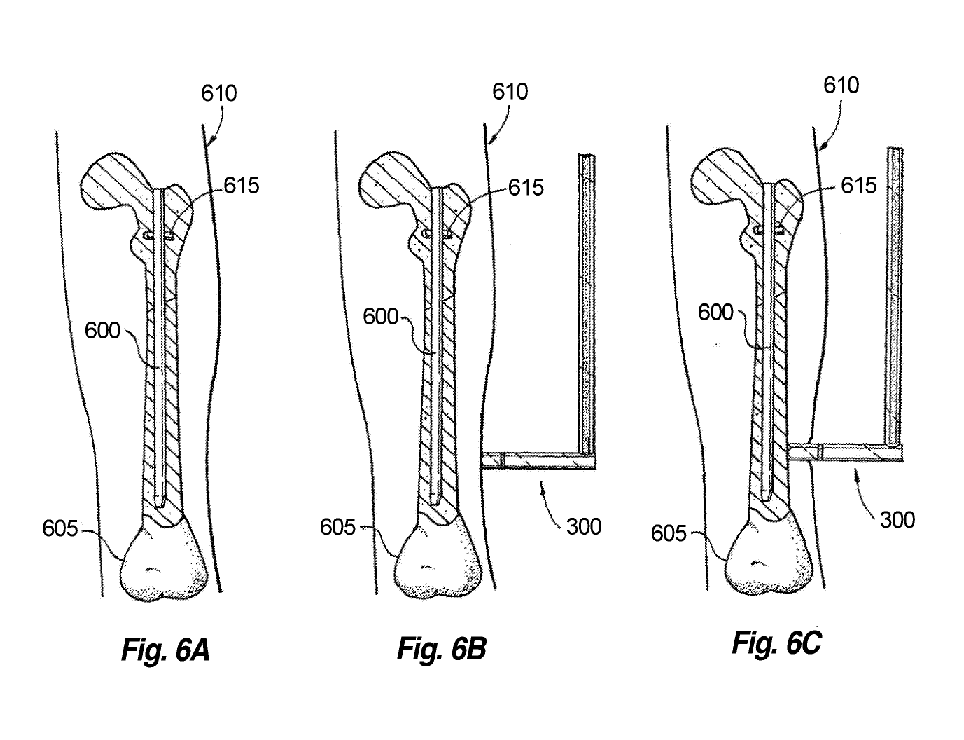

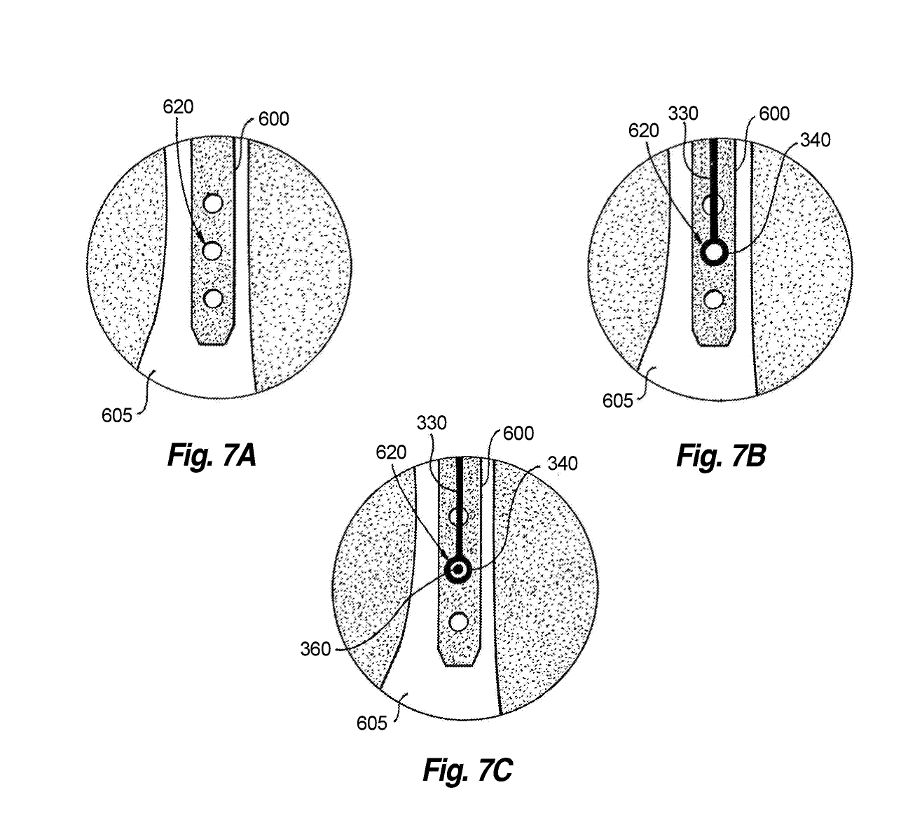

So formed, the fixture 300 becomes an extremely useful tool for locating and drilling holes in long bones for distal locking screws. FIG. 5 shows a flow diagram of an illustrative method 500 of using the fixture 300 in such a manner, in accordance with an illustrative embodiment of the invention. Before the method is initiated, it is assumed that an IM nail 600 similar to the IM nail 100 has been inserted into the medullary canal of a femur 605 in a patient's leg 610, and a proximal locking screw 615 has already been installed. A diagrammatic representation of this condition is shown in FIG. 6A. It is further assumed that a c-arm x-ray machine has been positioned so that an image of a target distal locking hole 620 appears as a round circle on the machine's display. A diagrammatic representation of what might be seen on the display of the x-ray machine is shown in FIG. 7A. In the image, an image of the lower portion of the femur 605 and an image of the IM nail 600 are clearly visible. Since the IM nail 600 is radiopaque (e.g., formed of metal), its image is darker than the image of the surrounding femur 605. An image of the target distal locking hole 620 in the IM nail 600 is also clearly discernible. A round shape assures that the x-ray imaging is occurring normal to the target distal locking hole 620 and not oblique thereto.

C-arm-type x-ray machines (frequently simply called “c-arms”) will already be familiar to one having ordinary skill in the relevant arts, and are commercially available from several vendors. A c-arm utilizes fluoroscopic imaging to present visible images in essentially real time while utilizing imaging equipment that may be rotated about the patient to allow the patient to be imaged at various angles. The x-ray generator typically may operate either in a continuous or pulsed modes with continuous modes providing the greatest image resolution.

Step 505 of the method 500 involves the initial alignment of the fixture 300 (without the removable insert 315) with the target distal locking hole 620. During this step, the surgeon moves the fixture 300 so as to place the radiopaque band 340 in line with the target distal locking hole 620 on the x-ray machine's display. At the same time, the surgeon also lines up the long radiopaque rod 330 with the longitudinal axis of the IM nail 600. FIG. 6B shows a diagrammatic representation of the fixture 300 in relation to the femur 605 and the IM nail 600 during step 505. A diagrammatic representation of what the surgeon may see on the x-ray display upon achieving initial alignment is shown in FIG. 7B. An image of the radiopaque band 340 and an image of a portion of the long radiopaque rod 330 are now visible with the radiopaque band 340 overlying the target distal locking hole 620. Here again, it is important that the radiopaque band 340 appear round (rather than oblong) so as to assure proper alignment with the target distal locking hole 620.

It is noted that the unique shape of the fixture 300 allows a surgeon to accomplish the initial alignment in step 505 without exposure to radiation even while x-ray imaging is utilized to accomplish the desired alignment. One major advantage of the fixture 300 thereby becomes evident. With its extended length, the long section 305 of the fixture 300 provides an extended handle by which the surgeon can grasp and manipulate the fixture 300 while keeping his or her hands outside of the region being irradiated, even while the short section 310 still falls within the region of x-ray imaging. To avoid radiation in this manner, a surgeon performing step 505 may simply grasp the long section 305 well away from where it attaches to the short section 310.

It is further noted that, after accomplishing the initial alignment in step 505, the jagged edge 345 of the short section 310 of the fixture 300 is in contact with the patient's leg 610 (i.e., skin) (FIG. 6B). The jagged nature of the jagged edge 345 is intended to provide a higher coefficient of friction with the skin than would occur with a smoother surface. The jagged edge 345 thereby reduces the chance the fixture 300 will slip out of alignment both in step 505 and in the following steps where it ultimately comes in contact with the femur 605.

The next step, step 510, involves maintaining the positioning of the fixture 300 and inserting the removable insert 315 into the central channel 335 so as to confirm correct alignment of the fixture 300 relative to the target distal locking hole 620 by x-ray imaging. The complementary sets of threads 347, 357 are threadably engaged to hold the removable insert 315 in place, assuring that it doesn't fall out unintentionally. To again reduce exposure of the surgeon to radiation, the x-ray generator may be turned off while the removable insert 315 is inserted, and then turned back on. All the time, the surgeon may continue holding the fixture 300 outside of the region exposed to radiation. FIG. 7C shows a representation of what the surgeon may see on the x-ray display after insertion of the removable insert 315 in step 510 and with proper alignment of the fixture 300. An image of the long radiopaque rod 330, an image of the radiopaque band 340, and an image of the short radiopaque rod 360 are now visible with the image of the short radiopaque rod 360 appearing centered over the target distal locking hole 620 and appearing centered in the image of the radiopaque band 340. The image of the short radiopaque rod 360 appears round (rather than elongated), indicating proper normal alignment with the target distal locking hole 620 rather than an oblique alignment. With alignment confirmed, the removable insert 315 may be removed from the remainder of the fixture 300, again with the x-ray generator off so as to reduce the surgeon's exposure.

Step 515 involves making an incision in the patient's skin proximate to the jagged edge 345 of the short section 310 of the fixture 300, and then translating the fixture 300 such that the jagged edge 345 contacts the outer cortex of the femur 605. The situation shown in FIG. 6C is thereby created, where an end of the short section 310 is placed against the femur 605 (i.e., against the bone). The incision can be made in several different ways. For example, after alignment of the fixture 300, as represented in FIG. 6B, the jagged edge of the short section 310 may be pressed against the patient's skin with enough force to temporarily mark the skin. Subsequently, a conventional scalpel or other suitable surgical instrument may be used to create the required incision at that mark.

Alternatively, again with the fixture 300 in proper alignment as in FIG. 6B, a narrow knife with a relatively long handle may be inserted through the central channel 335 of the short section 310, and the knife made to penetrate past the skin of the patient to the outside cortex of the long bone (in this particular case, the femur 605). With the knife still placed against the bone and in the central channel 335, the fixture 300 may then be translated along the knife until the jagged edge 345 of the short section 310 also contacts the bone. The knife may then be removed to obtain FIG. 6C. In this manner, the fixture 300 initially acts as a guide for inserting the knife, and the inserted knife subsequently becomes a guide for translating the fixture 300 to the bone. If the surgeon so desires, a long cylindrical rod with a sharp tip, such as a Steinmann pin, may be passed through the central channel 335 in place of the knife. A Steinmann pin may be made to penetrate the soft tissue to the outside cortex of the bone with little more than a light push. If the opening in the skin made by a knife or Steinmann pin is insufficiently narrow, an incision started by the knife or Steinmann pin can be expanded with a scalpel.

During the translation from the arrangement in FIG. 6B to that in FIG. 6C, x-ray imaging may be utilized to maintain the alignment of the fixture 300 while the surgeon's hand or hands remain unexposed at the end of the long section 305. That is, during the translation, the surgeon may attempt to maintain an x-ray image similar to that shown in FIG. 7B. After placement in step 515, the surgeon may again insert the removable insert 315 into the central channel 335 in step 520 to confirm alignment by an x-ray image similar to that shown in FIG. 7C.

The fixture 300 is now positioned such that the central channel 335 of the short section 310 is aligned with the target distal locking hole 620. Accordingly, in step 525, a drill bit may be inserted through the central channel 335 of the short section 310, and the femur 605 drilled through to make an opening for a distal locking screw. Step 525 may be accomplished with the x-ray generator off, again affording the surgeon the ability to perform the procedure without x-ray exposure. While drilling the bone, the short section 310 shields the patient's soft tissue from exposure to the drill bit. With the hole drilled, the surgeon may then place the distal locking screw. Before drilling, a sharp pin such as a Steinmann pin may also optionally be passed through the central channel 335 and tapped into the femur 650 to start a hole.

It should again be emphasized that the above-described embodiments of the invention are intended to be illustrative only. Other embodiments can use different types and arrangements of elements for implementing the described functionality, or different method steps. These numerous alternative embodiments within the scope of the appended claims will be apparent to one skilled in the art.

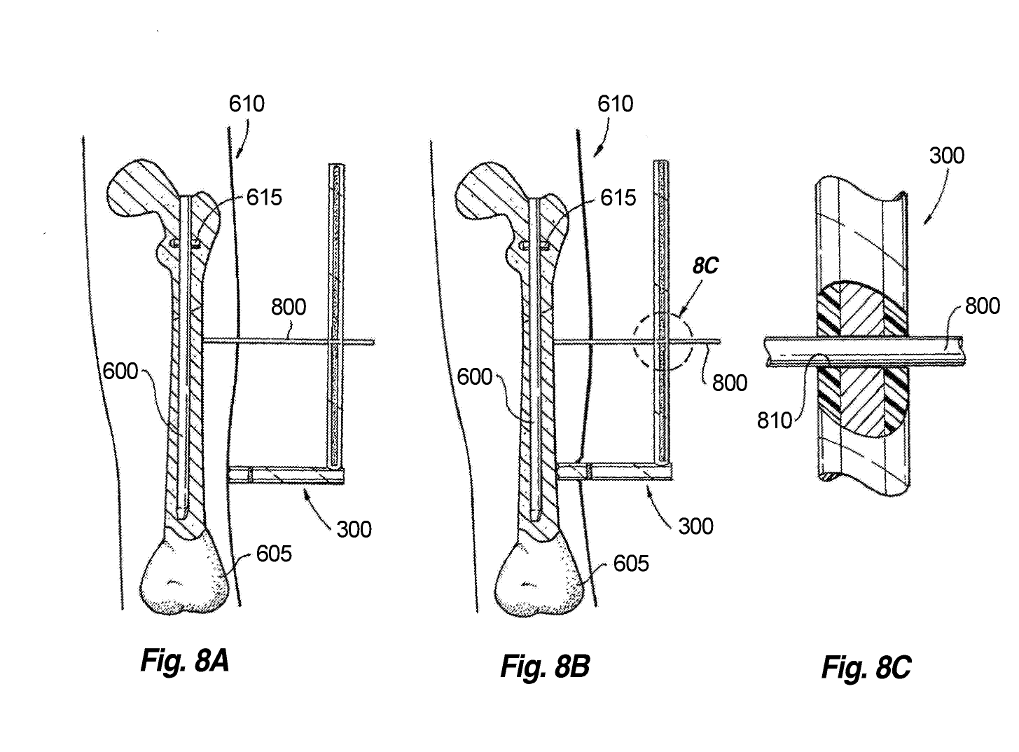

For example, in alternative embodiments falling within the scope of the invention, step 515 may also be implemented utilizing an additional middle stabilizing rod, as shown diagrammatically in FIGS. 8A and 8B. FIGS. 8A and 8B are duplicative of FIGS. 6B and 6C, respectively, but include a middle stabilizing rod 800, which passes transversely through a passage 810 in the long cylindrical body 320 and partially penetrates into, or abuts, the femur 605. FIG. 8C shows a magnified partial sectional view of the passage 810 in the long section 305. Proximate to the passage 810, there is a short break in the long radiopaque rod 330. The middle stabilizing rod 800 can be placed at the end of step 505. So placed, the middle stabilizing rod 800 can further stabilize the fixture 300 during steps 510-525, including helping to guide the fixture 300 while it is translated from external to the patient's leg 610 (FIG. 8A) to actually abutting the femur 605 (FIG. 8B) during step 515.

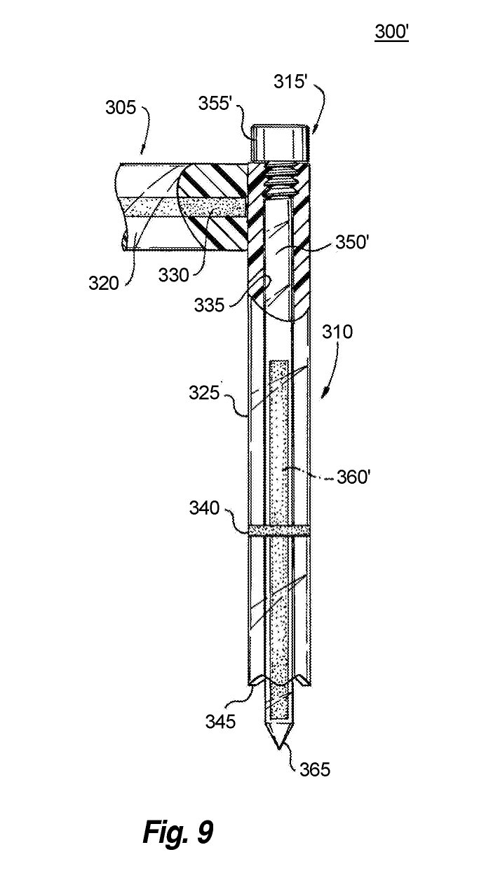

In even other embodiments, a longer and sharpened version of the removable insert 315 may be utilized. FIG. 9 shows a partial sectional view of a portion of the fixture 300 with a modified removable insert 315′ inserted and threaded therein. The modified removable insert 315′ includes a cap 335′, a cylindrical insert body 350′, a short radiopaque rod 360′, and a sharpened tip 365′. Unlike the removable insert 315 discussed above, the modified removable insert 315′ is long enough that an end of the removable insert opposite the cap 335′ is positioned outside the central channel 335 with the cap 335′ abutted against and threaded into the short section 310. The sharpened tip 365′ defines a form of cutting edge that allows the surgeon to use the modified removable insert 315′ to make an initial incision into the patient's leg at the beginning of step 515 of the method 500. This initial incision may subsequently be enlarged by the surgeon with a scalpel or other implement in order to make room to translate the fixture 300 to the bone in step 515.

While the long cylindrical body 320, the short cylindrical body 325, the long radiopaque rod 330, the central channel 335, and the short radiopaque rod 360 are cylindrical, alternative embodiments may utilize different shapes. For example, in one or more embodiments falling within the scope of the invention, the long cylindrical body and/or long radiopaque rod may be square tubular (i.e., have square cross-sections). Accordingly, it is reinforced that the particular shapes set forth in the embodiments above are intended to be illustrative and non-limiting.

The fixture 300 and its methods of use provide several advantages. In the method 500, for example, the combination of the long radiopaque rod 330, the radiopaque band 340, and the short radiopaque rod 360 provide a surgeon with an opportunity to easily and accurately align the fixture 300 with a target distal locking hole, all the while, with little or no exposure of the surgeon to x-ray radiation. That is, when the x-ray generator is activated, the fixture 300 affords the surgeon the ability to hold the fixture 300 outside of the beam of radiation. After the fixture 300 is properly positioned, the drilling may be accomplished with the x-ray generator off.

The fixture 300 also provides a surgeon with a means of avoiding the “guess and check” method of fixing a distal locking screw, substantially increasing efficiency and reducing the time needed to perform the procedure. The time that a patient is under anesthesia and exposed to x-rays is thereby reduced. At the same time, time spent in the operating room is reduced, which may allow a hospital to perform additional procedures in a given time.

Finally, the fixture 300, and more generally apparatus coming within the scope of the invention, may be used on any long bone and are therefore “universal.” The need to train surgeons on different jigs for different long bones is eliminated. The fixture 300, for example, may be used to place distal locking screws in a femur, tibia, and humerus without modification.

If the fixture 300 is made of plastic and metal as indicated above, it is contemplated that the fixture may be made disposable and shipped as a once-use, already-sterilized kit. The fixture 300 may also be offered in different sizes. As indicated in FIG. 6C, the short section 310 of the fixture 300 must be long enough to penetrate the skin of a patient and have the jagged edge 345 abut the outside cortex of the long bone being repaired. Larger patients may need fixtures with longer short sections.

All the features disclosed herein may be replaced by alternative features serving the same, equivalent, or similar purposes, unless expressly stated otherwise. Thus, unless expressly stated otherwise, each feature disclosed is one example only of a generic series of equivalent or similar features.

Any element in a claim that does not explicitly state “means for” performing a specified function or “step for” performing a specified function is not to be interpreted as a “means for” or “step for” clause as specified in AIA 35 U.S.C. §112(f). In particular, the use of “steps of” in the claims herein is not intended to invoke the provisions of AIA 35 U.S.C. §112(f).

What is claimed is:

1. An apparatus comprising:
a long section comprising a long radiopaque rod running along a long axis;
a short section joined to the long section and defining a central channel running therethrough along a short axis that is substantially normal to the long axis; and
a removable insert at least partially disposed inside the central channel and comprising a short radiopaque rod oriented along the short axis.

2. The apparatus of claim 1, wherein the short section further comprises a radiopaque band encircling a portion of the central channel.

3. The apparatus of claim 2, wherein the short radiopaque rod is cylindrical and the radiopaque band is circular.

4. The apparatus of claim 1, wherein the long section and the short section join in an L shape.

5. The apparatus of claim 1, wherein at least a portion of the short section is externally cylindrical.

6. The apparatus of claim 1, wherein:
the central channel defines internal threads;
the removable insert defines external threads; and
the internal threads are threadably engageable with the external threads.

7. The apparatus of claim 1, wherein the removable insert defines a cap at one end too large to fit into the central channel.

8. The apparatus of claim 1, wherein each of the long radiopaque rod and the short radiopaque rod comprises a metal.

9. The apparatus of claim 1, wherein an end of the short section defines a jagged edge.

10. The apparatus of claim 1, wherein the long section defines a passage passing therethrough transverse to the long axis.

11. The apparatus of claim 1, wherein:
the removable insert defines a cap at one end too large to fit into the central channel; and
an end of the removable insert opposite the cap is positioned outside the central channel with the cap abutted against the short section.

12. The apparatus of claim 11, wherein the end of the removable insert opposite the cap defines a cutting edge.

13. A method of placing a locking screw into a locking hole of an intramedullary nail in a bone, the method comprising the steps of:
obtaining an apparatus comprising:
a long section comprising a long radiopaque rod running along a long axis;
a short section joined to the long section and defining a central channel running therethrough along a short axis that is substantially normal to the long axis; and
a removable insert at least partially disposed inside the central channel, and comprising a short radiopaque rod oriented along the short axis; and
causing an image of the short radiopaque rod to appear centered over an image of the locking hole utilizing x-ray imaging.

14. The method of claim 13, wherein:
the short section further comprises a radiopaque band encircling a portion of the central channel; and
the step of causing the short radiopaque rod to appear centered over the locking hole utilizing x-ray imaging comprises causing an image of the short radiopaque rod to appear centered in an image of the radiopaque band via x-ray imaging.

15. The method of claim 13, further comprising the step of placing an end of the short section against the bone.

16. The method of claim 13, further comprising the step of drilling the bone with a drill bit inserted through the central channel.

17. The method of claim 13, further comprising the steps of:
passing a stabilizing rod through a passage in the long section; and
placing the stabilizing rod against, or partially into, the bone.

18. The method of claim 13, wherein the x-ray imaging comprises fluoroscopy.
